(19) United States
(12) Patent Application Publication
(10) Pub. No.: US 2014/0257322 A1
(43) Pub. Date: Sep. 11, 2014

(54) UTERINE MANIPULATOR

(71) Applicant: Gyrus ACMI, Inc., d.b.a., Olympus Surgical Technologies America, (US)

(72) Inventors: Kester J. Batchelor, Mound, MN (US); John C. Hanneman, Shakopee, MN (US)

(73) Assignee: Gyrus ACMI, Inc., d.b.a., Olympus Surgical Technologies America, Southborough, MA (US)

(21) Appl. No.: 13/793,630

(22) Filed: Mar. 11, 2013

(51) Int. Cl. A61B 17/42

(52) U.S. Cl. CPC A61B 17/4241
USPC 606/119

(57) ABSTRACT

A uterine manipulator for manipulation of a uterus of a patient is provided. The uterine manipulator has an articulation section having a proximal end and a distal end. The articulation section defines a channel therethrough. An elongate tensioning member extends through the channel of the articulation section. A tension release mechanism is connected to the tensioning member. The tension release mechanism is configured to selectively relax and tighten the tensioning member. A manipulator end is disposed at the distal end of the articulation section. The manipulator end is configured to be inserted into a vaginal canal and the uterus of the patient.

### FIELD

The present disclosure relates to a uterine manipulator, and more particularly, to a uterine manipulator for maneuvering, manipulating and/or clamping the uterus of a patient.

### BACKGROUND

The statements in this section merely provide background information related to the present disclosure and may or may not constitute prior art.

Uterine manipulators are used during surgical procedures to maneuver the uterus and/or cervix, for example, to allow safe access and visualization of various internal structures of the lower pelvis. Typical uterine manipulators are either static or have a means of motion via the device handpiece. Both the static and hand-operated variants can somewhat manipulate the uterus in relation to the body of the patient by using the manipulator pivoting at a point along its length to create a fulcrum for movement, or in the case of the hand operated variant, through levers actuated at the proximal end of the device to manipulate the tissues.

Static systems manipulate the uterus, for example, by pivoting against the patient's own tissue or anatomy. Examples of such static systems are shown in U.S. Patent Application Publication Nos. 2010/0106163 and 2008/0208210, which are herein incorporated by reference in their entireties. Examples of hand operated systems are shown in U.S. Pat. Nos. 5,643,285; 5,645,561; and 5,409,496, which are herein incorporated by reference in their entireties. These devices, however, fail to obtain a full range of manipulation without extensive and expensive to manufacture lever systems. These uterine manipulators have various levels of angular movement in a single plane. Further planes are only achieved by the user exerting and maintaining a twisting or lateral force upon the device handle. This can lead to user fatigue and loss of control of the tissues being manipulated. To overcome this, stabilizers have been developed that attach to the patient table to hold the manipulator in a set angle without requiring the user to maintain the fatiguing hold.

### SUMMARY

The present disclosure provides a uterine manipulator configured to increase the range of movement that may be locked in place.

In one aspect, which may be combined with or separate from the other aspects described herein, the present disclosure provides a uterine manipulator for manipulation of a uterus of a patient. The uterine manipulator includes an articulation section having a proximal end and a distal end. The articulation section defines a channel therethrough. An elongate tensioning member extends through the channel of the articulation section. A tension release mechanism is connected to the tensioning member. The tension release mechanism is configured to selectively relax and tighten the tensioning member. A manipulator end is disposed at the distal end of the articulation section. The manipulator end is configured to be inserted into a vaginal canal and the uterus of the patient.

In another aspect, which may be combined with or separate from the other aspects described herein, a uterine manipulator for manipulation of a uterus of a patient is provided. The uterine manipulator includes an elongate body having a proximal end and a distal end. The elongate body defines a channel therethrough. A tensioning member extends through the channel of the elongate body. The tensioning member is configured to be selectively tightened and relaxed. A manipulator end is disposed at the distal end of the elongate body. The manipulator end is configured to be inserted into a vaginal canal and the uterus of the patient. The elongate body is adjustable between a rigid state and a bendable state. The elongate body is in the rigid state when the tensioning member is tightened, and the elongate body is in the bendable state when the tensioning member is relaxed.

Accordingly, pursuant to one aspect of the invention, there is contemplated a uterine manipulator for manipulation of a uterus of a patient, the uterine manipulator comprising one or more of the following: an articulation section having a proximal end and a distal end, the articulation section defining a channel therethrough; an elongate tensioning member extending through the channel of the articulation section; a tension release mechanism connected to the tensioning member, the tension release mechanism configured to selectively relax and tighten the tensioning member; and a manipulator end disposed at the distal end of the articulation section, the manipulator end configured to be inserted into a vaginal canal and the uterus of the patient.

Accordingly, pursuant to another aspect of the invention, there is contemplated a uterine manipulator for manipulation of a uterus of a patient, the uterine manipulator comprising one or more of the following: an elongate body having a proximal end and a distal end, the elongate body defining a channel therethrough; a tensioning member extending through the channel of the elongate body, the tensioning member being configured to be selectively tightened and relaxed; and a manipulator end disposed at the distal end of the elongate body, the manipulator end configured to be inserted into a vaginal canal and the uterus of the patient, wherein the elongate body is adjustable between a rigid state and a bendable state, the elongate body being in the rigid state when the tensioning member is tightened and the elongate body being in the bendable state when the tensioning member is relaxed.

The invention may be further characterized by one or any combination of the features described herein, such as: the tension release mechanism is actuatable to relax the tensioning member to allow slack in the tensioning member; the articulation section comprises a plurality of articulatable segments; each articulatable segment of the plurality of articulatable segments is disposed adjacent to another articulatable segment of the plurality of articulatable segments; each articulatable segment has a cup-shaped convex portion and a receiving portion; the receiving portion of each articulatable segment is configured to receive the cup-shaped convex portion of an adjacent articulatable segment; each articulatable segment is moveable in an infinite number of directions with respect to an adjacent articulatable segment; each articulatable segment has multiple degrees of freedom with respect to an adjacent articulatable segment; the tension release mechanism is a spring-loaded button; the uterine manipulator further comprises a handle having a proximal end and a distal end, the distal end of the handle being disposed adjacent to the proximal end of the articulation section; the spring-loaded button is disposed at the proximal end of the handle; the uterine manipulator further comprises an annular body disposed around the articulation section for engaging a cervix of the patient; the uterine manipulator further comprises an inflatable membrane disposed on the manipulator end; the uterine manipulator further comprises a sealing member disposed adjacent to the annular body for sealing the uterine manipulator with respect to the vaginal canal of the patient; the tensioning member is a wire; the articulation section comprises a single joint connecting the manipulator end to a main body; the uterine manipulator further comprises a tension releasing part connected to the tensioning member; and the tension releasing part is actuatable to relax the tensioning member to allow slack in the tensioning member.

Further aspects, advantages and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1A is a perspective view of uterine manipulator in a first position, in accordance with the principles of the present disclosure;

FIG. 1B is a cross-sectional side view of the uterine manipulator of FIG. 1A, having another variation of an articulation segment, according to the principles of the present disclosure;

FIG. 2 is a perspective view of the uterine manipulator of FIGS. 1A-1B in a second position, in accordance with the principles of the present disclosure;

FIG. 3 is a perspective view of the uterine manipulator of FIGS. 1A-1B and 2 in a third position, according to the principles of the present disclosure;

FIG. 4 is a perspective view of the uterine manipulator of FIGS. 1A-1B, 2, and 3 in a fourth position, in accordance with the principles of the present disclosure;

FIG. 5 is a perspective view of an example of an articulatable segment of the uterine manipulator of FIGS. 1A-4, according to the principles of the present disclosure;

FIG. 6A is a perspective view of another example of an articulatable segment of the uterine manipulator of FIGS. 1A-4, in accordance with the principles of the present disclosure;

FIG. 6B is a perspective cross-sectional view of the articulatable segment of FIG. 6A, according to the principles of the present disclosure;

FIG. 7 is a perspective view of another uterine manipulator, in accordance with the principles of the present disclosure;

FIG. 8 is a perspective view yet another uterine manipulator, according to the principles of the present disclosure;

FIG. 9A is a side view of still another uterine manipulator, in accordance with the principles of the present disclosure; and

FIG. 9B is a side perspective view of the uterine manipulator of FIG. 9A, according to the principles of the present disclosure.

### DETAILED DESCRIPTION

The following description is merely exemplary in nature and is not intended to limit the present disclosure, application, or uses.

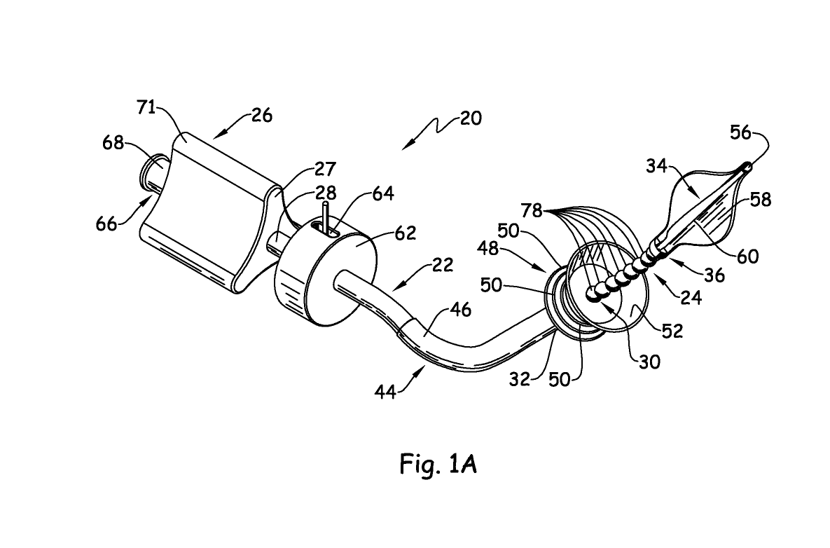

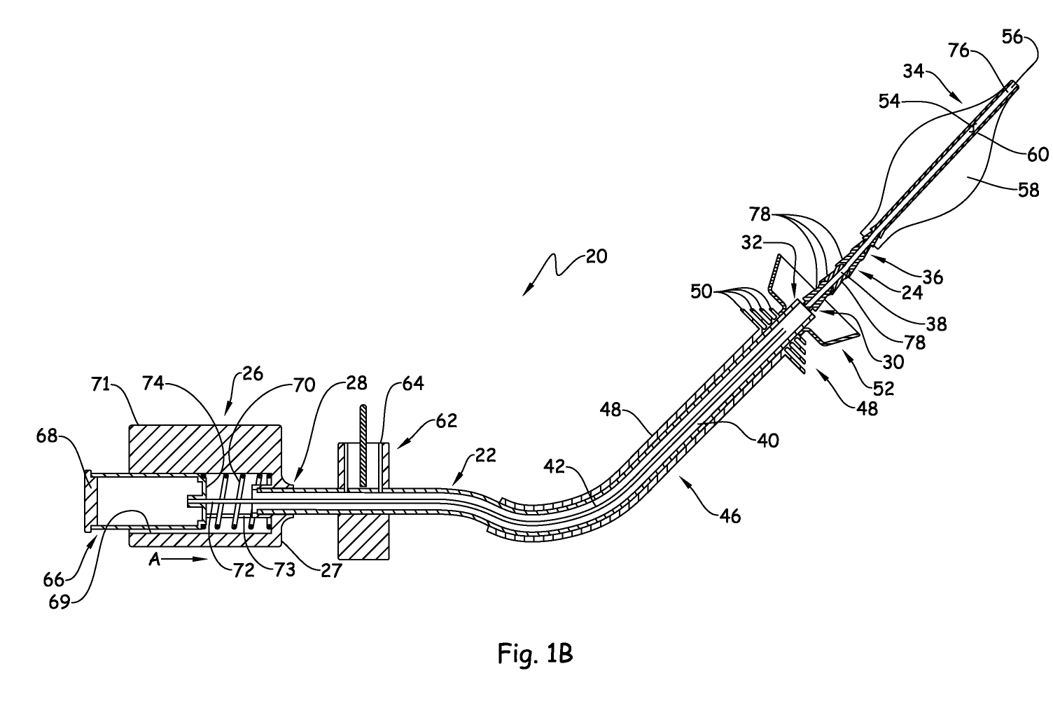

With reference to the figures, wherein like numerals indicate like components, and specifically with reference to FIGS. 1A-1B, an example of a uterine manipulator in accordance with the principles of the present disclosure is illustrated and generally designated at 20. The uterine manipulator 20 is intended to be inserted into a patient's uterus for maneuvering the uterus and/or cervix during various medical examinations and procedures, such as hysterectomies, oophorectomies, fertility studies, removal of tissue specimens, tumor removal, among other surgeries, by way of example. The uterine manipulator 20 may be inserted into the uterus through the vagina. The uterine manipulator 20 is used to manipulate, maneuver, position, hold, and/or visualize the uterus and/or the cervix during a surgery.

The uterine manipulator 20 has a main body portion 22 coupled to an articulation section 24. A handle 26 has a distal end 27 that is connected to a proximal end 28 of the main body portion 22. The articulation section 24 has a proximal end 30 that is coupled to a distal end 32 of the main body portion 22. Thus, the distal end 27 of the handle 26 is disposed on a proximal side of the proximal end 30 of the articulation section 24. A manipulator end 34 is attached to a distal end 36 of the articulation section 24. The manipulator end 34 is configured to be inserted into a vaginal canal and the uterus of the patient.

The articulation section 24 defines a channel 38 therethrough, and the main body portion 22 defines a channel 40 therethrough. In some variations, the articulation section 24 has the shape of an elongate body, or the articulation section 24 and the main body portion 22 combine to form an elongate body. The channel 38 of the articulation section 24 is in fluid communication with the channel 40 of the main body portion 22. An elongate tensioning member 42, such as a wire or string, extends through the channels 38, 40 of the articulation section 24 and the main body portion 22.

The uterine manipulator 20 may also have a sealing member 44 disposed around the main body portion 22. The sealing member 44 has a tubular portion 46 snuggly disposed over the main body portion 22, and a vaginal seal 48 (which may include a set of rings 50) configured to help seal the uterine manipulator 20 against the vaginal canal of the patient. In the addition, or in the alternative, the sealing member 44 may include a balloon, a closed cell foam pad, a reverse cup, or any other sealing mechanism known to those having ordinary skill in the art.

An annular body, such as a cup 52, may be disposed adjacent to and distally of the sealing member 46 for cutting against with a cutting instrument (not shown), or for engaging a cervix of the patient, by way of example.

In the illustrated embodiment of FIGS. 1A-1B, the manipulator end 34 is a tube having a channel 54 formed therethrough, and having an open end 56. The manipulator end 34 may have an inflatable membrane, such as a balloon 58 disposed thereon. The balloon 58 is shown deflated in FIGS. 1A-1B. In the illustrated embodiment, the balloon 58 is attached to the side 60 of the manipulator end 34, and not blocking the open end 56. The balloon 58 may act as an anchor for the uterine manipulator 20 when the balloon 58 is inflated, by way of example. In some variations, the balloon 58 could have spikes or arms extending therefrom (not shown) to assist in anchoring the balloon 58 to internal tissue.

Thus, an instrument, fluid, or medium could be introduced through the central channels 38, 40 of the main body portion 22 and the articulatable section 24 and into the patient's anatomy through the open end 56 of the manipulator end 34, in some embodiments. A boot 62 is attached to the main body portion 22 for introducing a member (not shown) into the channels 38, 40 of the uterine manipulator 20. The boot has an opening 64 in fluid communication with the channel 40 of the main body portion 22. A tube carrying dye or ink, for example, could be inserted into the channels 38, 40 and out of the open end 56 through the opening 64 of the boot 62.

A tension release mechanism 66 is connected to the tensioning member 42. The tension release mechanism 66 configured to selectively relax and tighten the tensioning member 42. In this embodiment, the tension release mechanism 66 is at least partially located within the handle 26.

The tension release mechanism 66, in this embodiment, is a press and release mechanism that may include a button 68 and one or more springs 70, such as a helical spring or another type of spring (e.g., leaf spring, Belleville spring), by way of example. The button 68 is configured to be pressed into the handle 26 to compress the spring 70. Thus, the button 68 is a spring-loaded button. More particularly, the handle 26 defines a cylindrical bore 69 in a proximal end 71 of the handle 26, and the button 68, having a complimentary cylindrical shape, slides into and out of the bore 69. A guide groove 73 may be formed in the handle 26 adjacent to and communicating with the bore 69 to guide the button 68 into and out of the bore 69.

The spring 70 is located in handle 26, but in other configurations, the spring 70 could be located in the channel 40 of the main body portion 22 or in the articulation section 24. In this embodiment, the button 68 is located at a proximal end 71 of the handle 26, but the button 68 could alternatively be located on another portion of the handle 68.

The button 68 is coupled to the tensioning member 42, and as such, when the button is pressed into the handle 62, the tension in the tensioning member 42 is released. More particularly, the tensioning member 42 has an end 72 that is connected to a distal end 74 of the button 68. A distal end 76 of the tensioning member 42 is connected to the manipulator end 34. When the button 68 is not compressed, in the deactivated position or rigid position, the tensioning member 42 is pulled tightly between the distal end 74 of the button 68 and the distal end 76 of the manipulator end 34. When the button 58 is pressed into the handle 68 in a proximal direction A, however, the end 72 of the tensioning member 42 is moved along the direction A, thus adding slack into the tensioning member 42. When the button 68 is released, the tensioning member 42 is pulled tightly again and regains tension. Thus, the tension release mechanism 68 is actuatable to relax the tensioning member 42 to allow slack in the tensioning member 42.

In the illustrated embodiment, the tension release mechanism 66 is shown including the spring-loaded button 68 and spring 70, but the tension release mechanism 66 could have additional or alternative components, such as a lever, one or more magnets, rotating tensioners (such as screw threads), bobbins (such as with a fishing reel-type tensioner), or solenoids, by way of example.

The uterine manipulator 20 is selectively lockable to hold any position to which the uterus is moved. The tensioning member 42 is selectively relaxed and tightened to allow movement of articulation section 24.

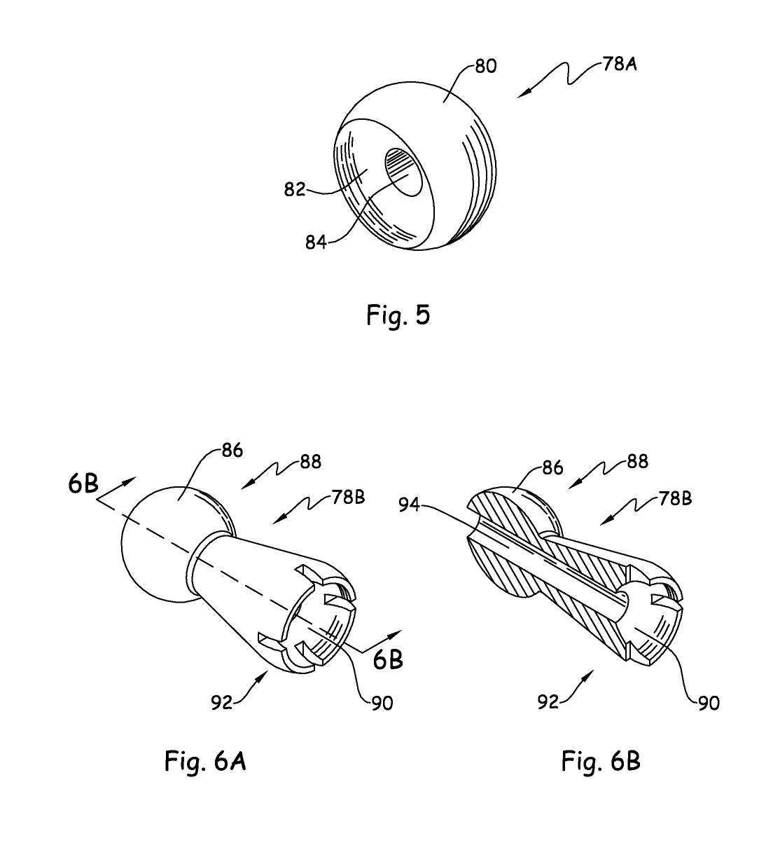

The articulation section 24 comprises a plurality of articulatable segments 78. In FIG. 1A, a first embodiment of the articulatable segments 78 is shown like the articulatable segments 78A in FIG. 5, which will be described in further detail below. In FIG. 1B, a second embodiment of the articulatable segments 78 is shown like the articulatable segments 78B in FIGS. 6A-6B, which will be described in further detail below.

Each articulatable segment 78 is disposed adjacent to another articulatable segment 78, and each articulatable segment 78 is connected to one or more adjacent articulatable segments 78, creating a serially connected link of articulatable segments 78. The articulatable segments 78 are each configured to move with respect to each other when the tensioning member 42 is in the relaxed or bendable state, which is when the tensioning member 42 is loosened and has slack, and is not tensioned; the tensioning member 42 is in the relaxed, bendable state when the tension release mechanism 66 is activated, e.g., when the button 68 is pushed into the handle 26. However, when the tension release mechanism 66 is deactivated (e.g., when the button 68 is not pressed), the tensioning member 42 is tightened and holds the articulatable segments 78 in place with respect to each other, which is the rigid state of the tensioning member 42.

Accordingly, the tension release mechanism 66 can be activated to move the articulatable segments 78 with respect to each other. Thus, the articulation section 24 is adjustable between the rigid state and the bendable state, the articulation section 24 being in the rigid state when the tensioning member 42 is tightened and the articulation section 24 being in the bendable state when the tensioning member 42 is relaxed. The uterine manipulator 20 is then moved around to the desired position, wherein the articulatable segments 78 move against each other and with respect to each other. To hold the articulatable segments 78 in place with respect to each other and with respect to the main body portion 22 and the manipulator end 34, the tension release mechanism 66 is released to tension the tensioning member 66.

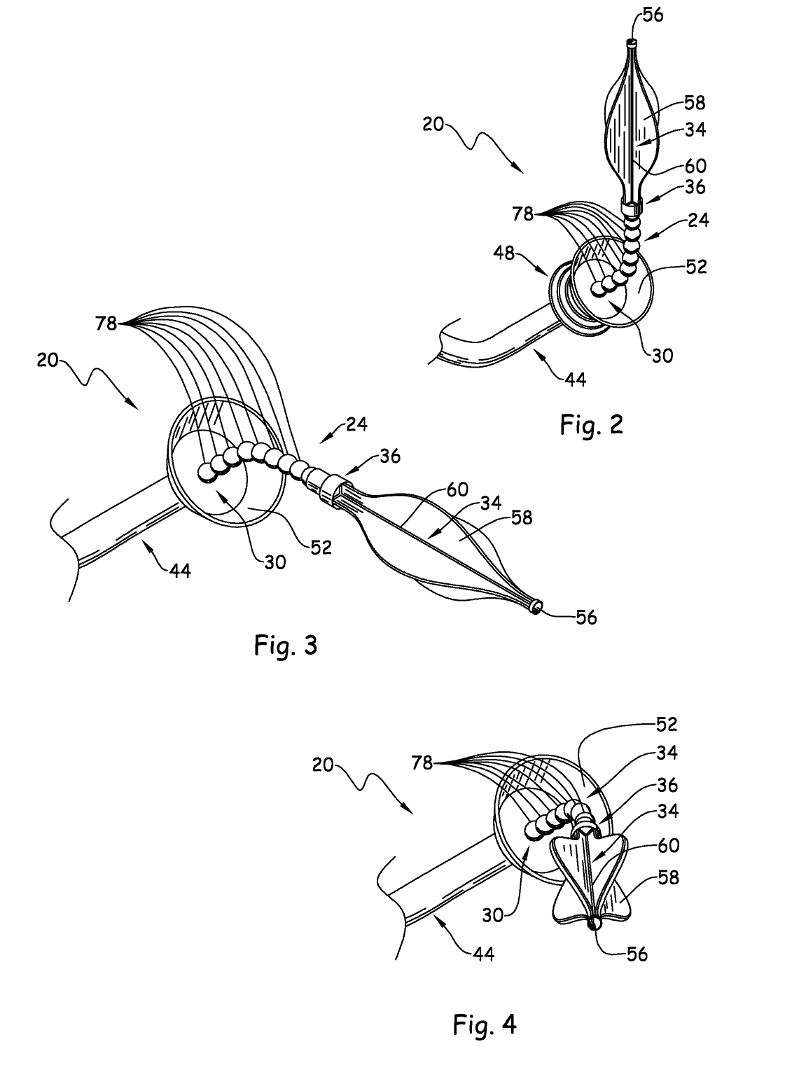

Referring to FIG. 2, the articulatable segments 78 have been moved with respect to each other and/or with respect to the main body portion 22 and/or the manipulator end 34, thus pointing the manipulator end 34 in a different direction than the direction that the manipulator end 34 was pointed in FIGS. 1A-1B. Thus, FIGS. 1A-1B show the uterine manipulator 20 in a first position, and FIG. 2 shows the uterine manipulator 20 in a second position. Likewise, FIGS. 3 and 4 show additional positions, third and fourth positions, respectively, of the uterine manipulator 20. Each articulatable segment 78 is moveable in an infinite number of directions with respect to an adjacent articulatable segment 78 and/or with respect to the main body portion 22 and/or the manipulator end 34. Each articulatable segment 78 has multiple degrees of freedom with respect to an adjacent articulatable segment 78 and/or with respect to the main body portion 22 and/or the manipulator end 34. With their cup-and-ball configuration, the articulatable segments 78 are movable along multiple planes with respect to each other and with respect to the main body portion 22 and/or the manipulator end 34. Therefore, the articulation section 24 can be maneuvered through the patient's inner anatomy, and selectively locked and released to do so.

Referring now to FIG. 5, an example of an articulatable segment 78 is illustrated and designated as 78A (and is shown in FIGS. 1A and 2-4. The articulatable segment 78A has a cup-shaped convex portion 80 and a concave complimentary cup-shaped receiving portion 82. The receiving portion 82 is configured to receive the cup-shaped convex portion 80 of an adjacent articulatable segment 78A. Thus, when the articulation section 24 is assembled, the articulatable segments 78 will form ball and socket joints with each other, wherein each adjacent articulatable segment 78 can move in an infinite number of planes with respect to each other and/or with respect to the main body portion 22 and/or the manipulator end. The main body portion 22 and/or the manipulator end 34 may also have the ball-and-socket configuration on an end to mate with the articulatable segments 78.

A channel 84 is formed in the center of the articulatable segment 78A, such that each channel 84 of the plurality of articulatable segments 78A is connected in fluid communication to form the channel 38 of the articulation section 24, described above. The tensioning member 42 and any ink tube or other insertable device (not shown) may extend through the channels 84.

Referring to FIGS. 6A and 6B, another example of an articulatable segment 78 is illustrated and designated as 78B, and is shown in FIG. 1B. The articulatable segment 78B is a more complex version of the articulatable segment 78, which has a convex cup-shaped ball 86 at a distal end 88 and an opening 90 at a proximal end 92. The opening 90 is sized to receive the ball 86 of an adjacent articulatable segment 78B. The proximal end 92 has portions forming slots 94 therein, to allow the proximal end 92 to snap over the ball 86 of an adjacent articulatable segment 78B, such that the adjacent articulatable segments 78B have a locking press-fit with respect to each other, which does not easily come apart but that allows the adjacent articulatable segments 78B to rotate with respect to each other and/or with respect to the main body portion 22 and/or the manipulator end 34, along the multiple and infinite degrees of freedom, as explained above.

A channel 94 is formed in the center of the articulatable segment 78B and extends from the opening 90 at the proximal end 92 to the distal end 88, such that each channel 94 of the plurality of articulatable segments 78B is connected in fluid communication to form the channel 38 of the articulation section 24, described above. The tensioning member 42 and any ink tube or other insertable device (not shown) may extend through the channels 94.

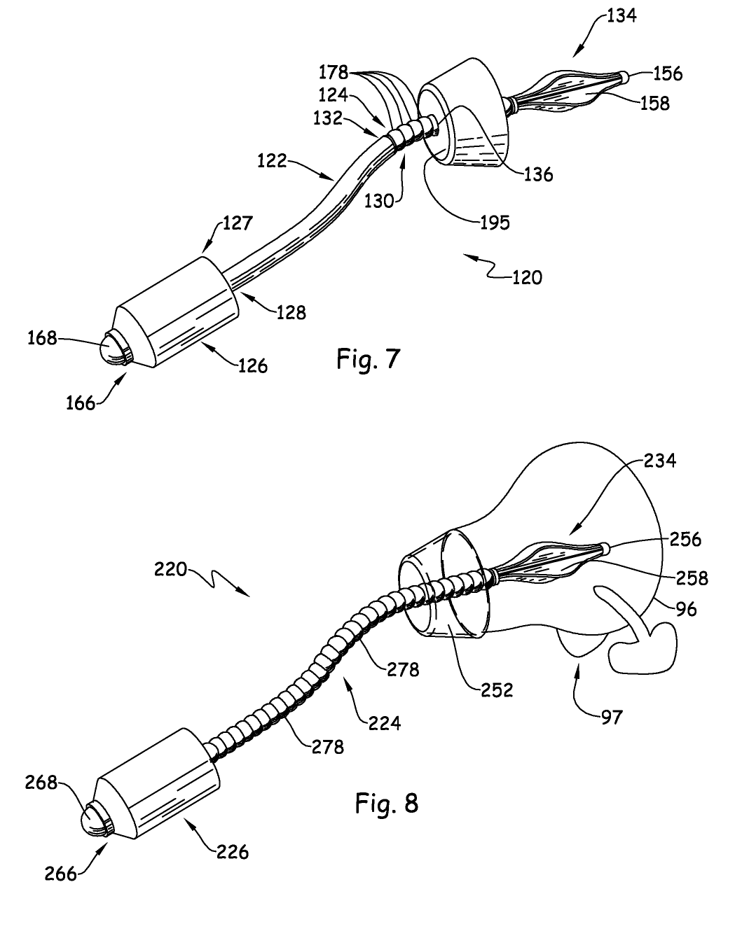

Referring now to FIG. 7, another variation of a uterine manipulator is illustrated and generally designated at 120. The uterine manipulator 120 may be used for the same purposes as the uterine manipulator 20 described above. Like the uterine manipulator 20, the uterine manipulator 120 shown in FIG. 7 has a main body portion 122 coupled to an articulation section 124. A handle 126 has a distal end 127 that is connected to a proximal end 128 of the main body portion 122. The articulation section 124 has a proximal end 130 that is coupled to a distal end 132 of the main body portion 122. A manipulator end 134 is coupled to a distal end 136 of the articulation section 124.

Like the uterine manipulator 20 described above, a channel (not shown) is defined through the main body portion 22, the articulation section 124, and the manipulator tip 134, through which a tensioning member (not shown) is disposed. Likewise, the handle 126 has a tension release mechanism 166 including a button 168 configured to tension and relax the tensioning member. The tensioning member and tension release mechanism 166 may be identical to, or varied from, those described above with respect to FIGS. 1A-1B, and such discussion is incorporated herein by reference. In the illustrated embodiment, the tension release mechanism 166 is shown including the spring-loaded button 168, but the tension release mechanism 166 could have additional or alternative components, as described above.

The uterine manipulator 120 may also have a sealing member (not shown) disposed around the main body portion 122, such as the sealing member 44 described above. An annular body, such as a cup 152, may be disposed adjacent to or around the distal end 136 of the articulation section 124 for cutting against with a cutting instrument (not shown), or for engaging a cervix of the patient, by way of example. The manipulator end 134 may be attached to the cup 152. In the embodiment of FIG. 7, the articulation section 124 is located on a proximal side 195 of the cup 152, rather than on a distal side of the cup as illustrated in FIGS. 1A-1B.

The manipulator end 134 may have an inflatable membrane, such as a balloon 158 disposed thereon, which may be the same or varied from the balloon 58 described above. An instrument, fluid, or medium could be introduced through the central channels of the main body portion 122 and the articulation section 124 and into the patient's anatomy through an open end 156 of the manipulator end 134. A boot (not shown) may also be attached to the main body portion 122 for introducing a member (not shown) into the central channels of the uterine manipulator 120. A tube carrying dye or ink, for example, could be inserted into the channels and out of the open end 156 through the boot (not shown).

The uterine manipulator 120 is selectively lockable to hold a position to which the uterus is moved. The tensioning member is selectively relaxed and tightened to allow movement of articulation section 124.

The articulation section 124 comprises a plurality of articulatable segments 178. The articulatable segments 178 may be similar or identical to one of the articulatable segments 78A, 78B shown in FIGS. 5, 6A, 6B, by way of example. Each articulatable segment 178 is disposed adjacent to another articulatable segment 178, and each articulatable segment 178 is connected to one or more adjacent articulatable segments 178, creating a serially connected link of articulatable segments 178.

The articulatable segments 178 are each configured to move with respect to each other and/or with respect to the main body portion 122 and/or the manipulator end 134 when the tensioning member is in the relaxed or bendable state, which is when the tensioning member is loosened and has slack, and is not tensioned. For example, the tensioning member could be in the relaxed, bendable state when the tension release mechanism 166 is activated, e.g., when the button 168 is pushed into the handle 126. However, when the tension release mechanism 166 is deactivated (e.g., when the button 168 is not pressed), the tensioning member is tightened and holds the articulatable segments 178 in place with respect to each other and with respect to the main body portion 122 and the manipulator end 134, which is the rigid state of the tensioning member.

Accordingly, the tension release mechanism 166 can be activated to move the articulatable segments 178 with respect to each other and/or with respect to the main body portion 122 and/or the manipulator end 134. Thus, the articulation section 124 is adjustable between the rigid state and the bendable state, the articulation section 124 being in the rigid state when the tensioning member is tightened and the articulation section 124 being in the bendable state when the tensioning member is relaxed. When the tensioning member is relaxed, the uterine manipulator 120 is moved around to the desired position, wherein the articulatable segments 178 move against each other and with respect to each other. To hold the articulatable segments 178 in place with respect to each other and/or with respect to the main body portion 122 and/or the manipulator end 134, the tension release mechanism 166 is released to tension the tensioning member.

Like the uterine manipulator 20 shown in FIGS. 1A-4, each articulatable segment 178 is moveable in an infinite number of directions and along multiple planes with respect to an adjacent articulatable segment 178 and/or with respect to the main body portion 122 and/or the manipulator end 134. Each articulatable segment 178 has multiple degrees of freedom with respect to an adjacent articulatable segment 178. Therefore, the articulation section 124 can be maneuvered through the patient's inner anatomy, and selectively locked and released to do so.

Referring now to FIG. 8, another variation of a uterine manipulator is illustrated and generally designated at 220. The uterine manipulator 220 may be used for the same purposes as the uterine manipulators 20, 120 described above. The uterine manipulator 220 has an articulation section 224 extending substantially along the entire length of the uterine manipulator 220. The articulation section 224 is connected to the handle 226 and the manipulator end 234, and the articulation section 224 extends between the handle 226 and the manipulator end 234.

Like the uterine manipulators 20, 120 described above, a channel (not shown) is defined through the articulation section 224, and the manipulator tip 234, through which a tensioning member (not shown) is disposed. Likewise, the handle 226 has a tension release mechanism 266 including a button 268 configured to tension and relax the tensioning member. The tensioning member and tension release mechanism may be identical to, or varied from, those described above with respect to the figures above, and such discussion is incorporated herein by reference.

The uterine manipulator 220 may also have a sealing member (not shown) disposed around the articulation section 224, such as the sealing member 44 described above. An annular body, such as a cup 252, may be disposed adjacent to or around the articulation section 224 for cutting against with a cutting instrument (not shown), or for engaging a cervix of the patient, by way of example.

The manipulator end 234 may have an inflatable membrane, such as a balloon 258 disposed thereon, which may be the same or varied from the balloons 58, 158 described above. An instrument, fluid, or medium could be introduced through the central channel of the articulation section 224 and into the patient's anatomy through an open end 256 of the manipulator end 234. A boot (not shown) may also be attached to the uterine manipulator 220, such as through the handle 226, for introducing a member (not shown) into the central channel of the uterine manipulator 220. A tube carrying dye or ink, for example, could be inserted into the channel and out of the open end 256 through the boot (not shown).

The uterine manipulator 220 is selectively lockable to hold any position to which the uterus is moved. The tensioning member is selectively relaxed and tightened to allow movement of the articulation section 224. The articulation section 224 comprises a plurality of articulatable segments 278. The articulatable segments 278 may be similar or identical to the articulatable segments 78A, 78B shown in FIGS. 5, 6A, 6B, by way of example. Each articulatable segment 278 is disposed adjacent to another articulatable segment 278, and each articulatable segment 278 is connected to one or more adjacent articulatable segments 278, creating a serially connected link of articulatable segments 278.

The articulatable segments 278 are each configured to move with respect to each other and/or with respect to the manipulator end 234 and/or the handle 226 when the tensioning member is in the relaxed or bendable state, which is when the tensioning member is loosened and has slack, and is not tensioned. For example, the tensioning member could be in the relaxed, bendable state when the tension release mechanism 266 is activated, e.g., when the button 268 is pushed into the handle 226. However, when the tension release mechanism 266 is deactivated (e.g., when the button 268 is not pressed), the tensioning member is tightened and holds the articulatable segments 278 in place with respect to each other and/or with respect to the manipulator end 234 and/or the handle 226, which is the rigid state of the tensioning member.

Accordingly, the tension release mechanism 266 can be activated to move the articulatable segments 278 with respect to each other and/or with respect to the manipulator end 234 and/or the handle 226. Thus, the articulation section 224 is adjustable between the rigid state and the bendable state, the articulation section 224 being in the rigid state when the tensioning member is tightened and the articulation section 224 being in the bendable state when the tensioning member is relaxed. When the tensioning member is relaxed, the uterine manipulator 220 may be moved around to the desired position, wherein the articulatable segments 278 move against each other and with respect to each other and/or with respect to the manipulator end 234 and/or the handle 226. To hold the articulatable segments 278 in place with respect to each other and/or with respect to the manipulator end 234 and/or the handle 226, the tension release mechanism 266 is released to tension the tensioning member. Like the uterine manipulators 20, 120 shown in the previous figures, each articulatable segment 278 is moveable in an infinite number of directions and along an infinite number of planes with respect to an adjacent articulatable segment 278 and/or with respect to the manipulator end 234 and/or the handle 226. Each articulatable segment 278 has multiple degrees of freedom with respect to an adjacent articulatable segment 278 and/or with respect to the manipulator end 234 and/or the handle 226. Therefore, the articulation section 224 can be maneuvered through the patient's inner anatomy, and selectively locked and released to do so.

In one form, the uterine manipulator 220 may be inserted into a uterus 96, by way of example. A pair of graspers 97 may be used to grasp and manipulate the uterus 96 while the tension release mechanism 266 is activated to put the uterine manipulator 220 into the bendable state. Once the uterus 96 is moved into the desired position, the tension release mechanism 266 may be released or deactivated to tension the tensioning member disposed in the channel of the articulation section 224 (see FIG. 1B and accompanying text for an example of the tensioning member that may be used here). The articulation section 224 is held, or locked, into place when the tension release mechanism 266 is released, thereby holding the uterus 96 in the desired position.

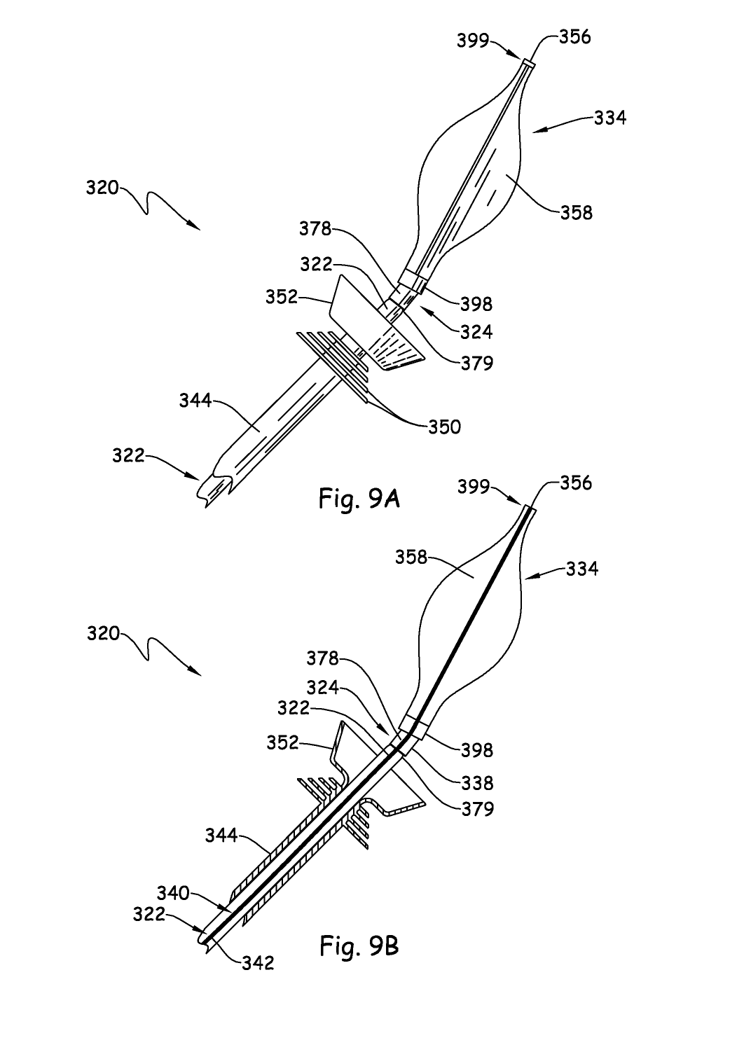

Referring now to FIGS. 9A-9B, yet another variation of a uterine manipulator is illustrated and generally designated at 320. The uterine manipulator 320 may be used for the same purposes as the uterine manipulators 20, 120, 220 described above. The uterine manipulator 320 has an articulation section 324 with only a single articulatable segment 378. The articulatable segment 378 is connected to the main body portion 322 and a proximal end 398 of the manipulator end 334. The articulatable segment 378 may be configured to rotate with respect to both the manipulator end 334 and the main body portion 322. In the alternative, the articulatable segment 378 may be fixed to one of the main body portion 322 and the manipulator end 334, such that the articulatable segment 378 only rotates or moves with respect to one of the manipulator end 334 and the main body portion 326. The articulatable segment 378 could be rigidly connected, or even formed unitarily with, the manipulator end 334 such that articulatable segment forms a single articulatable joint 379 between the manipulator end 334 and the main body portion 322.

Like the uterine manipulators hereinbefore described, the uterine manipulator 320 may have a handle (not shown) disposed at a proximal end (not shown) of the uterine manipulator 320. The main body portion 322 may extend between the handle (not shown) and the articulatable segment 378. Also like the uterine manipulators 20, 120, 220 described above, a channel 338 is defined through the articulation section 324, and a channel 340 is defined through the main body portion 322. The channels 338, 340 are in fluid communication with each other and with an opening 356 at the distal end 399 of the manipulator tip 334.

A tension release member 342 extends through the channels 338, 340 and is attached to a tension release mechanism (not shown) located in the handle (not shown) and to the manipulator end 334. The tension release mechanism may have any suitable configuration, such as the spring-loaded button hereinbefore described. The tensioning member and tension release mechanism may be identical to, or varied from, those described above with respect to the figures above, and such discussion is incorporated herein by reference.

The uterine manipulator 320 may also have a sealing member 344, including rings 350, disposed around the main body portion 322, like the sealing member 44 described above. An annular body, such as a cup 352, may be disposed around the main body portion 322 for cutting against with a cutting instrument (not shown), or for engaging a cervix of the patient, by way of example.

The manipulator end 334 may have an inflatable membrane, such as a balloon 358 disposed thereon, which may be the same or varied from the balloons 58, 158, 258 described above. An instrument, fluid, or medium could be introduced through the central channels 338, 340 of the articulation section 324 and the main body portion 322 and into the patient's anatomy through the open end 356 of the manipulator end 334. A boot (not shown) may also be attached to the uterine manipulator 320, such as through the handle (not shown) or the main body portion 322, for introducing a member (not shown) into the central channels 338, 340 of the uterine manipulator 320. A tube carrying dye or ink, for example, could be inserted into the channels 338, 340 and out of the open end 356 through the boot (not shown).

The uterine manipulator 320 is selectively lockable to hold any position to which the uterus is moved. The tensioning member 342 is selectively relaxed and tightened to allow movement of articulation section 324. The single articulatable segment 378 may be similar or identical to the articulatable segments 78A, 78B shown in FIGS. 5, 6A, 6B, by way of example.

The articulatable segment 378 is configured to move with respect to either or both of the manipulator end 334 and the main body portion 322 when the tensioning member 342 is in the relaxed or bendable state, which is when the tensioning member 342 is loosened and has slack, and is not tensioned. For example, the tensioning member 342 could be in the relaxed, bendable state when the tension release mechanism is activated, e.g., when the button is pushed into the handle. However, when the tension release mechanism is deactivated (e.g., when the button is not pressed), the tensioning member 342 is tightened and holds the articulatable segment 378 in place with respect to the main body portion 322 and/or the manipulator end 334, which is the rigid state of the tensioning member 342.

Accordingly, the tension release mechanism can be activated to move the articulatable segment 378 with respect to either or both of the main body portion 322 and the manipulator end 334. Thus, the articulation section 324 is adjustable between the rigid state and the bendable state, the articulation section 324 being in the rigid state when the tensioning member 342 is tightened and the articulation section 324 being in the bendable state when the tensioning member 342 is relaxed. The uterine manipulator 320 is then moved around to the desired position, wherein the articulatable segment 378 moves against and with respect to one or both of the main body portion 322 and the manipulator end 334. To hold the articulatable segment 378 in place with respect to the manipulator end 334 and/or the main body portion 322, the tension release mechanism is released to tension the tensioning member 342. Like the uterine manipulators 20, 120, 220 shown in the previous figures, the articulatable segment 378 is moveable in an infinite number of directions with respect to either or both of the manipulator end 334 and the main body portion 322. The articulatable segment 378 has multiple degrees of freedom with respect to at least one of the main body portion 322 and the manipulator end 334. Therefore, the articulation section 324 can be maneuvered through the patient's inner anatomy, and selectively locked and released to do so.

Each component of the uterine manipulator 20 may be formed of a biocompatible material. The main body portions 22, 122, 322, for example, may be formed of aluminum, a polymer, or Nitinol, such that the main body portion 322 may be twisted or slightly bent to maneuver through the patient's anatomy. In other words, the main body portions 22, 122, 322 may be at least partially flexible.

The description of the invention is merely exemplary in nature and variations that do not depart from the gist of the invention are intended to be within the scope of the invention. Such variations are not to be regarded as a departure from the spirit and scope of the invention. For example, variations in the various figures can be combined with each without departing from the spirit and scope of the present disclosure.

What is claimed is:

1. A uterine manipulator for manipulation of a uterus of a patient, the uterine manipulator comprising:
an articulation section having a proximal end and a distal end, the articulation section defining a channel therethrough;
an elongate tensioning member extending through the channel of the articulation section;
a tension release mechanism connected to the tensioning member, the tension release mechanism configured to selectively relax and tighten the tensioning member; and
a manipulator end disposed at the distal end of the articulation section, the manipulator end configured to be inserted into a vaginal canal and the uterus of the patient.

2. The uterine manipulator of claim 1, wherein the tension release mechanism is actuatable to relax the tensioning member to allow slack in the tensioning member.

3. The uterine manipulator of claim 2, wherein the articulation section comprises a plurality of articulatable segments.

4. The uterine manipulator of claim 3, wherein each articulatable segment of the plurality of articulatable segments is disposed adjacent to another articulatable segment of the plurality of articulatable segments, each articulatable segment having a cup-shaped convex portion and a receiving portion, the receiving portion being configured to receive the cup-shaped convex portion of an adjacent articulatable segment.

5. The uterine manipulator of claim 4, wherein each articulatable segment is moveable in an infinite number of directions with respect to an adjacent articulatable segment.

6. The uterine manipulator of claim 4, wherein each articulatable segment has multiple degrees of freedom with respect to an adjacent articulatable segment.

7. The uterine manipulator of claim 4, wherein the tension release mechanism is a spring-loaded button.

8. The uterine manipulator of claim 7, further comprising a handle having a proximal end and a distal end, the distal end of the handle being disposed on a proximal side of the proximal end of the articulation section, the spring-loaded button being disposed at the proximal end of the handle.

9. The uterine manipulator of claim 8, further comprising an annular body disposed around the articulation section for engaging a cervix of the patient.

10. The uterine manipulator of claim 9, further comprising an inflatable membrane disposed on the manipulator end.

11. The uterine manipulator of claim 10, further comprising a sealing member disposed adjacent to the annular body for sealing the uterine manipulator with respect to the vaginal canal of the patient.

12. The uterine manipulator of claim 2, wherein the tensioning member is a wire.

13. The uterine manipulator of claim 2, further comprising a main body portion connected to the articulation section, wherein the articulation section comprises a single joint connecting the manipulator end to the main body portion.

14. A uterine manipulator for manipulation of a uterus of a patient, the uterine manipulator comprising:
an elongate body having a proximal end and a distal end, the elongate body defining a channel therethrough;
a tensioning member extending through the channel of the elongate body, the tensioning member being configured to be selectively tightened and relaxed; and
a manipulator end disposed at the distal end of the elongate body, the manipulator end configured to be inserted into a vaginal canal and the uterus of the patient, wherein
the elongate body is adjustable between a rigid state and a bendable state, the elongate body being in the rigid state when the tensioning member is tightened and the elongate body being in the bendable state when the tensioning member is relaxed.

15. The uterine manipulator of claim 14, further comprising a tension releasing part connected to the tensioning member, the tension releasing part being actuatable to relax the tensioning member to allow slack in the tensioning member.

16. The uterine manipulator of claim 15, wherein the elongate body comprises a plurality of articulatable segments, each articulatable segment of the plurality of articulatable segments being disposed adjacent to another articulatable segment of the plurality of articulatable segments, each articulatable segment having a cup-shaped convex portion and a receiving portion, the receiving portion being configured to receive the cup-shaped convex portion of an adjacent articulatable segment.
